(19) United States
(12) Patent Application Publication
(10) Pub. No.: US 2020/0407689 A1
(43) Pub. Date: Dec. 31, 2020

(54) METHOD FOR CULTURING BONE MARROW-DERIVED MESENCHYMAL STEM CELLS

(71) Applicants: YAMAGUCHI UNIVERSITY, Yamaguchi-shi (JP); SHIBUYA CORPORATION, Kanazawa-shi (JP)

(72) Inventors: Isao SAKAIDA, Ube-shi (JP); Taro TAKAMI, Ube-shi (JP); Chikako TAMARI, Kanazawa-shi (JP)

(21) Appl. No.: 16/911,756

(22) Filed: Jun. 25, 2020

(30) Foreign Application Priority Data
Jun. 27, 2019 (JP) 2019-120099

(51) Int. Cl. C12N 5/0775; C12N 5/077; C12N 5/00

(52) U.S. Cl. CPC C12N 5/0665; C12N 5/0654; C12N 5/0031; C12N 2500/90; C12N 2502/1394; C12N 2506/1353; C12N 2523/00; C12N 2500/30

(57) ABSTRACT

A method for culturing bone marrow-derived mesenchymal stem cells which is capable of efficiently culturing the cells while minimizing the influence of animal-derived serum by use of a serum-free medium. The method includes inoculating cells extracted from a bone marrow fluid to a culture vessel, and performing medium replacement a plurality of times before initial passage, wherein in the inoculation, a medium supplemented with animal-derived serum is used, and in any of the plurality of times of medium replacement, the medium is exchanged with a serum-free medium without the use of the serum, followed by culture in the serum-free medium.

### BACKGROUND OF THE INVENTION

### Field of the Invention

The present invention relates to a method for culturing bone marrow-derived mesenchymal stem cells. Specifically, the present invention relates to a method for culturing bone marrow-derived mesenchymal stem cells using a serum-free medium.

### Description of the Related Art

In today's regenerative medicine field, mesenchymal stem cells are used in treatment. Such mesenchymal stem cells can be obtained by culturing cells extracted from a bone marrow fluid.

The bone marrow-derived mesenchymal stem cells are adherent cells, and a general culture method therefor involves inoculating cells extracted from a bone marrow fluid (mainly, bone marrow mononuclear cells) together with a medium to a culture vessel, and continuing culture with the medium replaced several times until the cells adhere to the culture vessel and proliferate stably.

Then, it is confirmed that the cells have proliferated sufficiently as mesenchymal stem cells, and passage was carried out thereafter such that the mesenchymal stem cells are detached from the culture vessel and distributed to a plurality of culture vessels. Then, the cells are cultured in each culture vessel with medium replacement and passage performed so that the mesenchymal stem cells proliferate into a necessary amount.

The medium for use in the culture of such mesenchymal stem cells is generally a basal medium supplemented with animal-derived serum such as bovine serum. The serum contains a component or the like promoting proliferation which cannot be made up for by the basal medium, whereas the risk of infection cannot be denied when cells cultured with the serum are used in treatment.

To cope with such a risk, a method involving using a serum-free medium from the inoculation of cells to a culture vessel, and a method involving using a medium supplemented with serum at the time of inoculation and then exchanging the medium with a serum-free medium for passage are disclosed as serum-free culture methods using a serum-free medium without the use of the animal-derived serum (Japanese Patent Laid-Open No. 2007-77).

In this context, comparative methods A1 and A2 shown in FIG. 1 depict experimental results about the culture of mesenchymal stem cells performed on the basis of the methods disclosed in Japanese Patent Laid-Open No. 2007-77, and depict experimental results of inoculating cells extracted from a bone marrow fluid, and then counting the number of cells obtained before initial passage.

Of these methods, the comparative method A1 employed, as a medium, a basal medium supplemented with bovine serum as animal-derived serum, and the comparative method A2 employed a serum-free medium without the use of the serum. As is evident from the comparison between these comparative methods A1 and A2, the number of obtained cells was larger by use of the medium supplemented with serum than by use of the serum-free medium; thus, the cell proliferation was promoted.

Specifically, according to these experimental results, use of the medium supplemented with animal-derived serum from inoculation to initial passage can be presumed to be more effective than use of the serum-free medium. However, the risk of infection still remains because the animal-derived serum is used.

In light of such results, the present invention provides a method for culturing bone marrow-derived mesenchymal stem cells which is capable of efficiently culturing the cells while minimizing the influence of animal-derived serum by use of a serum-free medium.

### SUMMARY OF THE INVENTION

Specifically, the method for culturing bone marrow-derived mesenchymal stem cells according to the invention of claim 1 is a method for culturing bone marrow-derived mesenchymal stem cells, comprising inoculating cells extracted from a bone marrow fluid to a culture vessel, and performing medium replacement a plurality of times before initial passage, the method characterized in that in the inoculation, a medium supplemented with animal-derived serum is used, and in any of the plurality of times of medium replacement, the medium is exchanged with a serum-free medium without the use of the serum, followed by culture in the serum-free medium.

According to the invention described above, the medium supplemented with serum is exchanged with a serum-free medium in any of the plurality of times of medium replacement performed before initial passage, whereby the bone marrow-derived mesenchymal stem cells can be efficiently cultured while the influence of animal-derived serum is minimized.

### BRIEF DESCRIPTION OF THE DRAWINGS

FIG. 1 is a table showing results of a first experiment and showing results about the number of cells in primary culture from inoculation to initial passage.

FIG. 2 is a table showing results of a second experiment and showing results about the number of cells through primary culture and expansion culture.

FIG. 3 is a table showing results of a third experiment and showing results about the number of cells in primary culture from inoculation to initial passage.

### DETAILED DESCRIPTION OF THE PREFERRED EMBODIMENTS

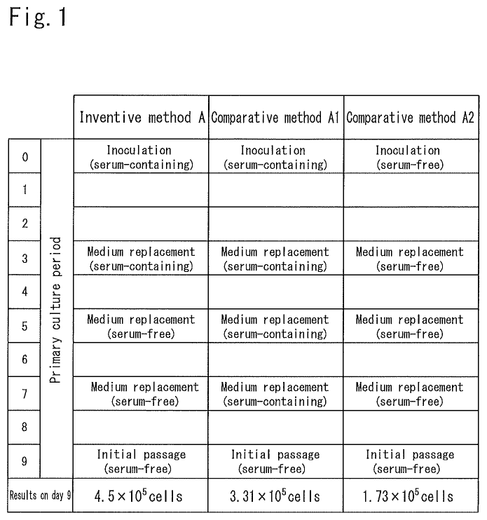

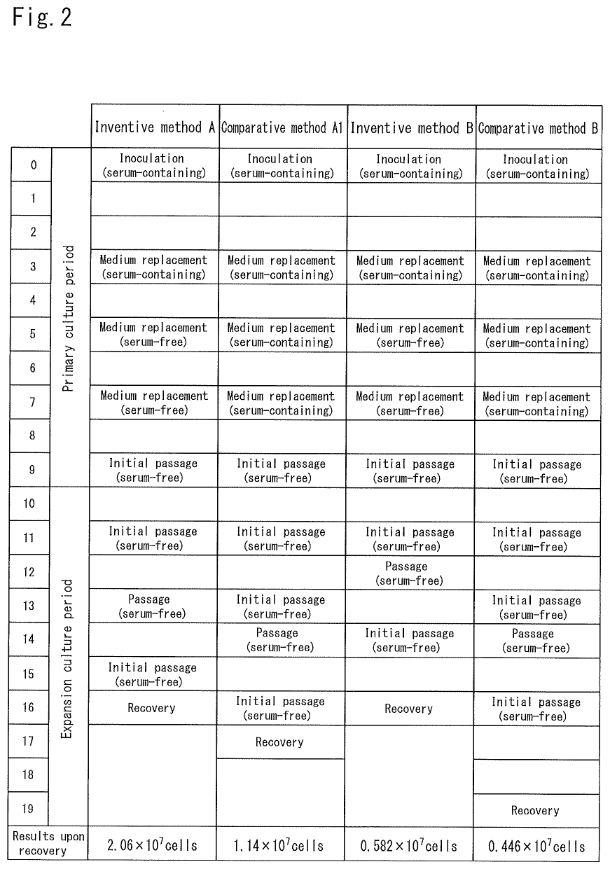

Hereinafter, the method for culturing bone marrow-derived mesenchymal stem cells according to the present invention will be described. FIGS. 1 and 2 are tables showing an inventive method according to the present invention and comparative methods including a conventional culture method performed for comparison therewith, according to the number of days of operation required for their culture operations.

Mesenchymal stem cells can be isolated from various tissues. The bone marrow-derived mesenchymal stem cells can be obtained relatively easily by culturing bone marrow mononuclear cells extracted from a bone marrow fluid.

A general method for culturing such mesenchymal stem cells is as mentioned in the section Description of the Related Art. More specifically, for inoculation, bone marrow mononuclear cells extracted from a bone marrow fluid are mixed with a medium and dispensed as a suspension to culture vessels. For medium replacement, the used medium is aspirated, and adherent mesenchymal stem cells are allowed to proliferate while a fresh medium is supplied. For passage, after confirmation of the degree of proliferation, the mesenchymal stem cells adhering to the bottom of the culture vessel are detached, washed, and mixed with a medium to prepare a suspension, which is then distributed and inoculated to a plurality of fresh culture vessels.

In this context, the culture to be performed after inoculation of the bone marrow mononuclear cells extracted from a bone marrow fluid to a culture vessel and before initial passage is defined as primary culture, and its period is referred to as a primary culture period. The culture of mesenchymal stem cells distributed to a plurality of culture vessels by initial passage is defined as expansion culture, and its period is referred to as an expansion culture period.

The bone marrow fluid used in the present experiment was purchased for tests. Each operation is generally carried out in one day without spanning days, though the time zone differs depending on a situation.

For the culture of the mesenchymal stem cells which are adherent cells, it is desirable that the inoculated cells should adhere rapidly to a culture vessel and start to proliferate in primary culture. When comparative methods A1 and A2 in FIG. 1 are compared, the medium supplemented with bovine serum as in the comparative method A1 exhibits nearly twice the proliferative properties in the serum-free medium as in the comparative method A2 in primary culture.

The basal medium has no cell adhesion factor. The addition of bovine serum to the basal medium promotes the adhesion performance of the cells. Meanwhile, the serum-free medium also has no adhesion factor. Thus, the adhesion performance is made up for by coating the surface of the culture vessel with an adhesion factor in advance or by adding an adhesion factor to the serum-free medium.

Specifically, the difference in proliferative properties in primary culture is probably due to difference in the adhesion performance of the cells. As for the comparative method A2, the adhesion factor used does not exert adherence properties equal to or higher than those of the serum; thus, presumably, all the cells do not adhere rapidly or proliferate.

As for the adhesion performance of cells, a component of animal-derived serum such as bovine serum still remains to be fully elucidated. Thus, artificial reproduction of the adhesion performance has not yet been achieved. Hence, animal-derived serum must be used for obtaining high cell adherence properties, whereas the risk of infection cannot be completely removed.

Accordingly, in the present invention, unprecedented marked effects can be found with the aim of attaining efficient culture for a shorter period while minimizing the utilization of animal-derived serum.

In this context, although the highest adhesion performance is required immediately after inoculation, the own ability of the cells to adhere is improved as proliferation proceeds. In addition, the serum-free medium is reinforced with a component effective for cell proliferation and exhibits higher proliferative performance than that for use of serum. On the basis of these ideas, unlike a culture method disclosed in Japanese Patent Laid-Open No. 2007-77 which involves exchanging a medium supplemented with serum with a serum-free medium at the time of initial passage, culture is performed in a basal medium supplemented with animal-derived serum for a predetermined period from inoculation, and the medium is exchanged with a serum-free medium at the time of any of a plurality of times of medium replacement performed before initial passage.

Hereinafter, inventive method A and comparative methods A1 and A2 shown in FIG. 1 will be described in detail. In the experiment shown in FIG. 1, inoculation was performed on day 0 of operation, and initial passage was performed on day 9 of operation. The number of mesenchymal stem cells obtained for the same primary culture period was compared among the methods.

In the present experiment, the number of mesenchymal stem cells in a culture vessel of 490 mm used in the experiment was counted on day 9 of operation, and used as experimental results.

In this context, the same serum-free medium was used in the inventive method A and the comparative methods A1 and A2 in FIG. 1. Also, the medium for inoculation in the inventive method A and the comparative methods A1 was prepared by adding the same bovine serum to the same basal medium. A generally available adhesion factor was added to the serum-free medium.

The inventive method A employs a medium supplemented with bovine serum from inoculation, and in the first medium replacement 3 days after the inoculation, the same medium supplemented with bovine serum as the removed medium is supplied to the culture vessel.

In the second medium replacement on day 5 of operation 2 days later therefrom, the medium supplemented with bovine serum is removed, and instead, a serum-free medium is supplied to the culture vessel to exchange the medium.

In the third medium replacement operation on day 7 of operation 2 days later therefrom, the serum-free medium is removed, and the same serum-free medium thereas is supplied to the culture vessel. The number of cells is counted for initial passage scheduled on day 9 of operation 2 days later therefrom.

Specifically, in the inventive method A, the used medium supplemented with bovine serum was exchanged with a serum-free medium in the second medium replacement among three times of medium replacement performed in the primary culture period.

As a result, the number of mesenchymal stem cells by the inventive method A was 4.5×10⁵ cells.

On the other hand, the comparative method A1 continuously employed a medium supplemented with bovine serum in a primary culture period from inoculation on day 0 of operation to initial passage.

As a result, the number of mesenchymal stem cells by the comparative method A1 was 3.31×10⁵ cells.

The comparative method A2 continuously employed a serum-free medium in a primary culture period from inoculation on day 0 of operation to initial passage.

As a result, the number of mesenchymal stem cells by the comparative method A2 was 1.73×10⁵ cells.

From the experimental results shown in FIG. 1, the inventive method A obtained approximately 1.36 times larger than the number of cells in the comparative method A1 and was thus able to be confirmed to produce the best results.

In the inventive method A, the cells probably adhered favorably to a culture vessel immediately after inoculation by continuously using the medium supplemented with bovine serum from days 0 to 4 of operation from inoculation to the second medium replacement.

Furthermore, the cells that have already adhered were presumably able to efficiently proliferate in the serum-free medium having high culture efficiency by exchanging the medium with the serum-free medium in the second medium replacement on day 5 of operation, and then culturing the cells in the serum-free medium.

By contrast, the comparative method A1 presumably resulted in a smaller number of cells than that of the inventive method A, because the medium supplemented with bovine serum having poor culture efficiency was continuously used even after sufficient adhesion of the cells.

Next, the experiment shown in FIG. 2 compared the number of mesenchymal stem cells obtained in primary culture as well as subsequent expansion culture between the inventive method A and the comparative method A1 in FIG. 1.

In this experiment, an experiment was conducted as inventive method B and comparative method B in which primary culture was performed by the same procedures as in the inventive method A and the comparative method A1, whereas the serum-free medium used differed from the serum-free medium used in the inventive method A and the comparative method A1.

In the experiment of FIG. 2, medium replacement, passage and cell recovery were each performed with the culture state observed in expansion culture following the primary culture. For cell counting at the time of recovery, the number of cells contained in 10 ml of a liquid was measured.

For the expansion culture following the primary culture in the inventive method A, medium replacement was performed on day 11 of operation 2 days after initial passage. Passage was performed on day 13 of operation 2 days later therefrom, and medium replacement was performed on day 15 of operation 2 days later therefrom. The cells were recovered on day 16 of operation following the day.

As a result, 2.06×10⁷ cells were confirmed.

By contrast, in the comparative method A1, the number of times of medium replacement was larger by 1, and the date of recovery was later by 1 day (day 17 of operation). In addition, the number of recovered cells was 1.14×10⁷ cells, which were smaller than that of the inventive method A.

Specifically, according to the present experimental results, the inventive method A was confirmed to be able to efficiently culture the cells in a shorter period than that of the comparative method A1.

The inventive method B employed a medium supplemented with bovine serum from inoculation to the second medium replacement 5 days later and performed culture using a serum-free medium, albeit different from that used in the inventive method A, in the subsequent initial passage and expansion passage, as in the inventive method A.

As a result, recovery was performed on day 16 of operation, as in the inventive method A, and 0.582×10⁷ cells were confirmed.

By contrast, the comparative method B performed culture using a medium supplemented with bovine serum from inoculation to initial passage, as in the comparative method A1, though the same serum-free medium as that of the inventive method B was used.

As a result, in the comparative method B, recovery was performed on day 19 of operation which was later by 3 days than that of the inventive method B, and the number of recovered cells was 0.446×10⁷ cells which fell below the results about the inventive method B.

According to the experimental results shown in FIG. 2, the comparison between the inventive method A and the comparative method A1 was able to reveal that the number of recovered cells in the inventive method A was approximately 1.8 times larger, in spite of the fact that the date of recovery in the inventive method A was earlier by 1 day.

Likewise, the comparison between the inventive method B and the comparative method B was also able to reveal that the number of recovered cells in the inventive method B was approximately 1.3 times larger, in spite of the fact that the date of recovery in the inventive method B was earlier by 3 days.

When the inventive method A and the inventive method B are compared, both the inventive methods enhanced proliferation as compared with the comparative methods, though the enhancement in proliferation was larger in the serum-free medium used in the inventive method A. This revealed that use of the culture method of the present invention produces similar effects even when any serum-free medium is used.

These results demonstrated that the culture method of the present invention (inventive method A) exerts higher proliferative properties than those of comparative method A1 in primary culture and further elevates the proliferative properties in expansion culture. This is presumably influenced by exchange with a serum-free medium before initial passage.

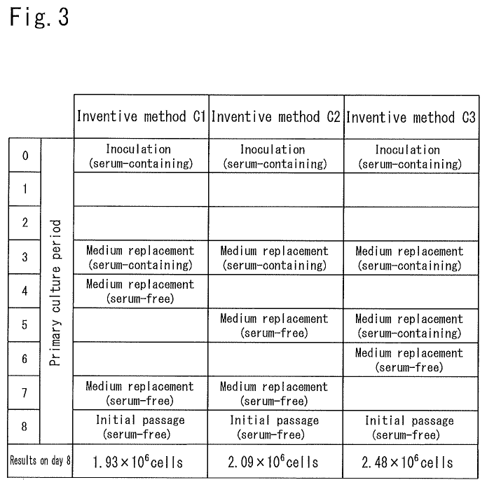

Next, the experiment shown in FIG. 3 compared the amount of mesenchymal stem cells recovered in initial passage when a medium supplemented with bovine serum was exchanged with a serum-free medium for medium replacement on different days of operation.

First, for comparison with the inventive method A shown in FIGS. 1 and 2, inventive method C2 employed a medium supplemented with bovine serum in medium replacement on day 3 of operation, exchanged the medium with a serum-free medium in the second medium replacement on day 5 of operation, and further performed the third medium replacement on day 7 of operation.

With respect to this inventive method C2, inventive method C1 performed the second medium replacement earlier (on day 4 of operation).

On the other hand, inventive method C3 performed the second medium replacement on day 5 of operation. However, a medium supplemented with bovine serum was used in this medium replacement. The medium was exchanged with a serum-free medium in the third medium replacement performed on day 6 of operation 1 day later therefrom.

In the present experiment, the number of mesenchymal stem cells in a culture vessel of ϕ90 mm used in the experiment was counted on day 8 of operation, and used as experimental results. The same medium supplemented with bovine serum and serum-free medium were used among the inventive methods.

In this context, the third medium replacement was performed on day 7, i.e., the day before initial passage, in the inventive methods C1 and C2, whereas one day was set between the third medium replacement and initial passage in the inventive method C3.

As a result of the experiment, 1.93×10⁶ mesenchymal stem cells were obtained in the inventive method C1; 2.09×10⁶ mesenchymal stem cells were obtained in the inventive method C2; and 2.48×10⁶ mesenchymal stem cells were obtained in the inventive method C3.

When the experimental results are compared, the numbers of cells obtained in the inventive methods C1 and C2 were at the same level, whereas the inventive method C3 resulted in a slightly larger number of obtained cells.

These experimental results shown in FIGS. 1, 2 and 3 will be discussed. Use of a medium supplemented with serum in inoculation and its replacement with the same medium supplemented with serum in the subsequent first medium replacement are probably effective, as in the inventive method in each experiment.

In this context, the inventive methods C1 and C2 shown in FIG. 3 produced similar results, demonstrating that use of the medium supplemented with serum for at least 3 days after inoculation is effective.

In the comparative method A1 shown in FIGS. 1 and 2, culture was performed in a medium supplemented with serum for medium replacement performed in a primary culture period, and the medium was exchanged with a serum-free medium for initial passage. In this case, the cells are cultured in an environment brought about by a medium different from the preceding one at a stage where the cells are detached from the culture vessel and inoculated to a fresh culture vessel.

By contrast, in the inventive methods, the medium was exchanged with a serum-free medium at a stage of medium replacement performed before initial passage. Therefore, in initial passage, the cells are inoculated to a fresh culture vessel containing the same medium as the preceding one. Thus, the passage presumably has a little influence on the cells.

In this case, the earliest possible exchange with a serum-free medium having high proliferative performance seems to be effective. However, referring to the results about the inventive methods C2 and C3 shown in FIG. 3, the presence of at least one day before initial passage (inventive method C2: 2 days between the second medium replacement and initial passage, inventive method C3: 1 day between the third medium replacement and initial passage) is effective for the period of culture in the serum-free medium.

1. A method for culturing bone marrow-derived mesenchymal stem cells, comprising inoculating cells extracted from a bone marrow fluid to a culture vessel, and performing medium replacement a plurality of times before initial passage, wherein
in the inoculation, a medium supplemented with animal-derived serum is used, and
in any of the plurality of times of medium replacement, the medium is exchanged with a serum-free medium without the use of the serum, followed by culture in the serum-free medium.

2. The method for culturing bone marrow-derived mesenchymal stem cells according to claim 1, wherein the cells are cultured in the medium supplemented with animal-derived serum for at least 3 days after the inoculation.

3. The method for culturing bone marrow-derived mesenchymal stem cells according to claim 1, wherein the cells are cultured in the serum-free medium for at least 1 day before the initial passage.
